(19) United States
(12) Patent Application Publication
(10) Pub. No.: US 2026/0294796 A1
(43) Pub. Date: Oct. 1, 2026

(54) OPHTHALMIC COOLING SOLUTIONS

(71) Applicant: Cayman Chemical Company Inc., Ann Arbor, MI (US)

(72) Inventors: Thushara KARUNASEKARA, Ann Arbor, MI (US); Shannon STACEY, Canton, MI (US); Mitchell DE LONG, Chapel Hill, NC (US)

(21) Appl. No.: 19/629,565

(22) Filed: Mar. 26, 2026

Related U.S. Application Data
(60) Provisional application No. 63/778,038, filed on Mar. 26, 2025.

(51) Int. Cl. A61K 9/00; A61K 9/08; A61K 31/045; A61K 31/167; A61K 47/02; A61K 47/14; A61K 47/18; A61K 47/26; A61K 47/44

(52) U.S. Cl. CPC A61K 9/0048; A61K 9/08; A61K 31/045; A61K 31/167; A61K 47/02; A61K 47/14; A61K 47/186; A61K 47/26; A61K 47/44

(57) ABSTRACT

The present disclosure provides an ophthalmic composition comprising cooling agents; saline or one or more of demulcents, disinfectants or surfactants ingredient; and about 0.01% weight/volume to about 10% weight/volume of a tonicity agent.

### CROSS REFERENCE TO RELATED APPLICATIONS

This application claims the benefit of priority under 35 U.S.C. § 119(e) of U.S. Provisional Application No. 63/778,038, filed Mar. 26, 2025, the disclosure of which is incorporated by reference herein in its entirety.

### BACKGROUND

Ophthalmic compositions are needed which provide a cooling sensation when artificial tears, also known as tear substitutes or other ocular wetting or restoring solutions are needed, and for contact lens solutions. These formulations, characterized as water-based, ocularly dosed formulations lacking any active pharmaceutical ingredient other than a demulcent, can be irritating or otherwise uncomfortable and thus a cooling sensation is needed to reduce discomfort upon administration and thereafter. Typically, these ophthalmic agents are administered as solutions for both ease of dosing and reduced discomfort upon administration. However, often artificial tears and the like are not sufficiently tolerated as dosed. In particular, those who have dry or irritated eyes might find that these solutions fail to bring immediate relief, or may in fact be irritating themselves. Therefore, compositions that create an environment where by saline, artificial tears, and rewetting or soaking solutions may be delivered with a cooling sensation created that minimizes discomfort upon administration is needed. It has surprisingly been discovered that the major disadvantages of the use of a single cooling ingredient can be overcome by the specific combination of two or more cooling agents.

### SUMMARY

In one aspect, the present disclosure provides ophthalmic compositions for the treatment of an ophthalmic disease, condition or disorder, in a subject in need thereof, the composition comprising: menthol and/or WS-12; and about 0.0001% weight/volume to about 5.0% weight/volume of one or more demulcents, saline, disinfectants or surfactants; and about 0.01% weight/volume to about 10% weight/volume of a tonicity agent.

In a second aspect, the present disclosure provides methods and kits for the treatment of an ophthalmic disease, condition or disorder, in a subject in need thereof, the kit comprising: one or more containers, the ophthalmic composition of claim 1, and instructions for use of the ophthalmic composition.

### DETAILED DESCRIPTION

In an aspect, the present disclosure provides an ophthalmic composition comprising one or more cooling agents from about 0.0001% weight/volume to about 0.01% weight/volume of a solution; and about 0.0001% weight/volume to about 1% weight/volume of one or more cooling agent(s). In another aspect, the present disclosure provides an ophthalmic composition comprising one or more demulcents, disinfectants or surfactants from about 0.001% weight/volume to about 5.0% weight/volume of a solution; and about 0.0001% weight/volume to about 1% weight/volume of two or more cooling agent(s). In another aspect, the present disclosure provides an ophthalmic composition comprising one or more of the specific cooling agents menthol and WS-12. The IUPAC name of WS-12 as its single most active diastereomer and enantiomer is: (1R,2S,5R)-N-(4-methoxyphenyl)-5-methyl-2-propan-2-ylcyclohexane-1-carboxamide.

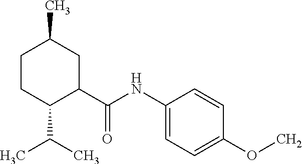 [SMILES: COc1ccc(NC(=O)C2C[C@H](C)CC[C@H]2C(C)C)cc1]

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The conjunctive term “or” includes any and all combinations of one or more listed elements associated by the conjunctive term. For example, the phrase “an apparatus comprising A or B” may refer to an apparatus including A where B is not present, an apparatus including B where A is not present, or an apparatus where both A and B are present. The phrases “at least one of A, B, . . . and N” or “at least one of A, B, . . . N, or combinations thereof” are defined in the broadest sense to mean one or more elements selected from the group comprising A, B, . . . and N, that is to say, any combination of one or more of the elements A, B, . . . or N including any one element alone or in combination with one or more of the other elements which may also include, in combination, additional elements not listed.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

For the purposes of the present disclosure, the phrase “artificial tears” and/or the phrase “tear substitutes” and/or the phrase “contact lens solutions” refer to solutions without recognized APIs present other than one or more demulcents. This includes all of the commercial formulations that are marketed for such purposes such as tear insufficiency. This list includes, but is not limited to:
Artificial Tears and Lubricants
Thera Tears® brand products
Refresh® brand products (including Refresh Tears®, Refresh Plus®, Refresh Optive®, Refresh Repair®, Refresh Optive Advanced®)
GenTeal® brand products
Soothe® brand products
Oasis® brand products
Blink® tears brand products
Systane® brand products
Akorn® artificial tears brand products
Murine® tears brand products
FreshKote® brand products
Dry Eye solutions
Lipiflow brand products
TearCare brand products
iLux brand products
Mibo Thermoflo brand products
EyeXpress brand products
BlephEx brand products
ITN TrueTear brand products
Multipurpose Solutions:
Opti-Free PureMoist Disinfecting Solution
Bausch+Lomb Biotrue Multi-Purpose Solution
Opti-Free Replenish
GoodSense Multi-Purpose Saline Solution
Leader Contact Multi-Purpose Lens Solution
Peroxide Solutions:
Clear Care Plus with HydraGlide
Saline Solutions:
Bausch+Lomb Sensitive Eyes Saline Solution
PuriLens Mini Preservative Free
Other Solutions:
Boston Advance Contact Lens Solution
Boston Simplus Contact Lens Solution
Optimum by Lobob Wetting and Rewetting Drops
Clerz Plus Contact Lens Drops
Bausch+Lomb re'nu Advanced Formula
Bausch+Lomb re'nu MultiPlus
Alcon branded: OptiFree and Clear Care.

### Compositions

In an aspect, the present disclosure provides an ophthalmic composition comprising WS-12 and/or menthol. WS-12 and/or menthol may be present in an amount of about 0.00001% w/v to about 0.06% w/v. Suitably, WS-12 and/or menthol may be present in an amount of about 0.001% w/v to about 0.03%. When present together, the molar ratio of WS-12 to menthol shall be no more than about 100:1 to no less than about 1:100. For the purposes of this invention, the phrase “menthol” shall include (−) menthol, (+) menthol, racemic menthol, and borneol, in all mixtures and proportions. The phrase “WS-12” shall include its enantiomers and diastereomers in all proportions, as well as the other members of the WS family of cooling agents, including but not limited to the longer lasting ones WS-5 and WS-10. WS-23 may also be used, particularly in combination with WS-12 and menthol when three cooling agents are used. The IAPAC name of WS-12 as its single most active diastereomer and enantiomer is: (1R,2S,5R)-N-(4-methoxyphenyl)-5-methyl-2-propan-2-ylcyclohexane-1-carboxamide. The IUPAC name for (−)-menthol, its single most active enantiomer and diastereomer is: (1R,2S,5R)-5-methyl-2-propan-2-ylcyclohexan-1-ol.

### Buffers

Suitable buffers include, but are not limited to, acetic acid, citric acid, carbonic acid, phosphoric acid, boric acid, the pharmaceutically acceptable salts thereof, and tromethamine. The buffer may be present in an amount of from about 0.01% weight/volume to about 1% weight/volume or about 0.1% w/v to about 0.9% w/v, or about 0.3% w/v to about 0.8% w/v, or about 0.4% w/v to about 0.6% w/v. Suitably, the buffer may be present in at least 0.01% w/v, at least 0.05% w/v, at least 0.1% w/v, at least 0.3% w/v, or at least 0.5% w/v. Suitably, the buffer may be present in an amount of no more than 1.0% w/v, no more than 0.8% w/v, no more than 0.6% w/v, or no more than 0.5% w/v.

### Tonicity Agents

The tonicity, or osmolality, of the product can be adjusted to hypotonicity, isotonicity, or hypertonicity relative to normal tears by use of conventional materials known in the art. Tonicity agents are typically ionic compounds. Examples of tonicity agents include, but are not limited to, sodium chloride, potassium chloride, mannitol, dextrose, glycerine, and propylene glycol. Suitably, the tonicity agent may be present in an amount of from about 0.01% weight/volume to about 10% weight/volume or about 1% w/v to about 10% w/v, or about 2.5% w/v to about 7.5% w/v, or about 4% w/v to about 6% w/v. Suitably, the tonicity agent may be present in at least 0.01% w/v, at least 0.05% w/v, at least 1% w/v, at least 2.5% w/v, at least 4% w/v, or at least 5% w/v. Suitably, the tonicity agent may be present in an amount of no more than 10% w/v, no more than 7.5% w/v, no more than 6% w/v, or no more than 5% w/v.

### Other Components

In embodiments, the composition may also contain pH adjusting agents in an amount sufficient to adjust the pH of the composition to between about 4.5 to about 7.5. Suitably, the composition has a pH of about 4.5 to about 7.2 or about 4.5 to about 7.1. Suitable pH adjusting agents include, but are not limited to, sodium hydroxide.

In embodiments, the composition may also contain an emulsifying agent. Suitable emulsifying agents include, but are not limited to, polyoxyl 40 stearate, polyethoxylated castor oil, and combinations thereof, and other agents known to one skilled in the art.

Ophthalmic products are typically packaged in unit dose or multidose form. Preservatives may be present to prevent or inhibit microbial contamination. Suitable preservatives include, but are not limited to, benzalkonium chloride, thimerosal, chlorobutanol, methyl paraben, propyl paraben, phenylethyl alcohol, edtate disodium, boric acid, sorbic acid, or other agents known to one skilled in the art. If present, the preservative may be present in an amount of about 0.001% w/v to about 1.0% w/v, or about 0.01% w/v to about 1.0% w/v, or about 0.1% w/v to about 1.0% w/v. Suitably, the preservative may be present in at least 0.001% w/v, at least 0.005% w/v at least 0.01% w/v, at least 0.05% w/v, at least 0.1% w/v, or at least 0.5% w/v. Suitably, the preservative may be present in an amount of no more than 1.0% w/v, no more than 0.9% w/v, no more than 0.5% w/v, or no more than 0.1% w/v.

Other components commonly used in ophthalmic compositions, such as surfactants, comfort-enhancing agents, solubilizing agents, antioxidants, and stabilizing agents, may also be present.

### Formulations and Methods of Use

The ophthalmic formulations of the present disclosure may be formulated in various dosage forms suitable for topical ophthalmic delivery, including solutions, suspensions, emulsions, and gels. The compositions are suitably aqueous.

The methods comprise topically applying to the affected eye(s) of the subject in need of treatment, for example, a human subject, a non-human mammal, (for example, a research animal used in ophthalmic disease research, (for example, a primate, a mouse, a rat, a ferret, a gerbil, a guinea pig, a rabbit, a goat, a pig), a domestic dog or a cat; a beneficial or therapeutically effective amount of a composition according to the present disclosure. The frequency and amount of dosage can be readily determined by one of skill in the art based on various clinical factors. The methods typically comprise topical application of one or two drops once or twice a day, but as needed may be up to about 12 times a day.

### EXAMPLES

The present disclosure has multiple aspects, illustrated by the following non-limiting examples. In the various examples, the below materials and characterization techniques have been used.

### Example: Formulation with WS-12 and Menthol

Table 1 shows a representative example of an eye care solution.

The solution was prepared in a 500 mL volumetric flask according to the following steps:

### Combining Ingredients:

A 500 mL volumetric flask containing 300 mL of water was heated to 45° C. on a hotplate. The required amounts of Benzalkonium Chloride, Boric Acid, Chlorobutanol, Edetate Disodium, Potassium Aspartate, and Sodium Chloride were quantitatively transferred into the volumetric flask. The solution was stirred at 300 rpm at 45° C. with a magnetic stir bar until complete dissolution.

### Preparing Cremophor RH40, WS-12, and Menthol Mixture:

Cremophor RH40 was heated to 35° C. to melt, and the required amount was weighed into a scintillation vial containing the prescribed amount of WS-12 and menthol. The vial was capped and placed on another hotplate at 50° C. until WS-12 and menthol dissolved completely. While maintaining the stirring, this mixture was quantitatively transferred into the 500 mL volumetric flask. The scintillation vial was rinsed twice with warm water (50° C.) and transferred to the 500 mL volumetric flask.

### Dissolving Povidone:

The required amount of Povidone-K90 was gradually added to the 500 mL volumetric flask while maintaining the stirring at 300 rpm at 45° C. The stirring was increased to 600 rpm and kept until the Povidone-K90 was completely dissolved. Once the Povidone-K90 was completely dissolved, the stirring was lowered to 300 rpm and the temperature on the hotplate was turned off. The solution was diluted to approximately 495 mL and stirred until the foam was completely gone.

### Final Adjustments:

The solution and the stir bar were transferred to a beaker, and the pH was adjusted to ~7.0 with sodium borate solution at room temperature. The solution was transferred back to the flask and diluted to the mark with water. The flask was capped and gently turned upside down to mix. The final pH and the viscosity were measured at room temperature. The initial pH of the solution, prior to the addition of sodium borate, was approximately 5 and was adjusted to approximately 7 with a few drops of a 5 g/dL sodium borate solution.

TABLE 1

| Ingredient | Weight (mg) | Concentration (mg/mL) |
|---|---|---|
| WS-12 | 5.38 | 0.0108 |
| Menthol | 23.17 | 0.04634 |
| Povidone K90 | 10005.55 | 20.0111 |
| Cremophor RH40 | 7511.41 | 15.0228 |
| Benzalkonium Chloride | 50.85 | 0.1017 |
| Boric Acid | 50.32 | 0.1006 |
| Chlorobutanol | 101.01 | 0.20202 |
| Edetate Disodium | 50.74 | 0.1015 |
| Potassium Aspartate | 500.42 | 1.0008 |
| Sodium Chloride | 3755.36 | 7.51072 |
| Sodium Borate (5 g/dL) | N/A | N/A |

When an eyedrop of the above solution is delivered, the patient experiences a long-lasting cooling sensation without an initial sharp and possibly painful or irritating sensation.

It is understood that the foregoing detailed description and accompanying example are merely illustrative and are not to be taken as limitations upon the scope of the invention, which is defined solely by the appended claims and their equivalents.

1. An ophthalmic composition, comprising:
menthol and/or WS-12; and
about 0.0001% weight/volume to about 5.0% weight/volume of one or more demulcents, saline, disinfectants or surfactants; and
about 0.01% weight/volume to about 10% weight/volume of a tonicity agent.

2. The composition of claim 1, wherein the molar ratio of menthol to WS-12 is between 100:1 and 1:100 respectively.

3. The composition of claim 1, wherein the pH is from about 4.5 to about 7.5.

4. The composition of claim 1, further comprising a preservative.

5. The composition of claim 4, wherein the preservative comprises benzalkonium chloride.

6. The composition of claim 1, further comprising an emulsifying agent.

7. The composition of claim 6, wherein the emulsifying agent comprises polyoxyl 40 stearate, polyethoxylated castor oil, or a combination thereof.

8. The composition of claim 1, wherein the tonicity agent comprises mannitol.

9. The composition of claim 1, wherein the buffer comprises boric acid or its salts.

10. The composition of claim 1, further comprising water.

11. The composition of claim 1, further comprising a pH adjusting agent.

12. A method for treating an ophthalmic or otic condition or disorder to a subject in need thereof, the method comprising: administering a therapeutically effective amount of a composition comprising:
menthol and/or WS-12; and
about 0.0001% weight/volume to about 5.0% weight/volume of one or more demulcents, saline, disinfectants or surfactants; and
about 0.01% weight/volume to about 10% weight/volume of a tonicity agent.

13. The method of claim 12, wherein the molar ratio of menthol to WS-12 is between 100:1 and 1:100 respectively.

14. The method of claim 12, wherein the menthol is predominantly (−) menthol.

15. The method of claim 12, wherein the composition further comprising a preservative.

16. The method of claim 15, wherein the preservative comprises benzalkonium chloride.

17. The method of claim 12, wherein the subject is a human, a non-human mammal, a domestic dog or a cat.

18. A kit comprising, one or more containers, the ophthalmic composition of claim 1, and instructions for use of the ophthalmic composition.
